(19) United States
(12) Patent Application Publication
(10) Pub. No.: US 2015/0005621 A1
(43) Pub. Date: Jan. 1, 2015

(54) ULTRASONIC DIAGNOSTIC DEVICE AND CONTROL PROGRAM FOR THE SAME

(71) Applicant: GE MEDICAL SYSTEMS GLOBAL TECHNOLOGY COMPANY, LLC, Waukesha, WI (US)

(72) Inventor: Lei Liu, Tokyo (JP)

(73) Assignee: GE MEDICAL SYSTEMS GLOBAL TECHNOLOGY COMPANY, LLC, Waukesha, WI (US)

(21) Appl. No.: 14/310,426

(22) Filed: Jun. 20, 2014

(30) Foreign Application Priority Data
Jun. 27, 2013 (JP) 2013134647

(51) Int. Cl. A61B 8/08; A61B 8/00; A61B 5/06

(52) U.S. Cl. CPC A61B 8/0841; A61B 5/062; A61B 8/4254
USPC 600/424; 600/461

(57) ABSTRACT

An ultrasonic diagnostic device is provided. The ultrasonic diagnostic device includes an ultrasonic probe configured to acquire an echo signal by transmitting and receiving ultrasonic waves to and from a test object, a probe identification part configured to identify information about a position and orientation of the ultrasonic probe, a biopsy needle identification part configured to identify information about a position and orientation of a biopsy needle to be inserted into the test object, and a control part which controls at least one of two processes based on positional relations between the ultrasonic probe and the biopsy needle, the positional relations identified by the information from the probe identification part and the information from the biopsy needle identification part, wherein one of the two processes is the transmission and reception of the ultrasonic waves, and the other of the two processes is data processing based on the echo signal.

### CROSS REFERENCE TO RELATED APPLICATIONS

This application claims the benefit of Japanese Patent Application No. 2013-134647 filed Jun. 27, 2013, which is hereby incorporated by reference in its entirety.

### BACKGROUND OF THE INVENTION

The present invention relates to an ultrasonic diagnostic device used when a biopsy needle is inserted into a test object, and to a control program for use with the ultrasonic diagnostic device.

Ultrasonic diagnostic devices are capable of displaying an ultrasonic image of a test object in real time. Thus when a biopsy needle is to be inserted into the test object, the position of the biopsy needle can be verified using real time ultrasonic images (e.g., see Japanese Unexamined Patent Publication No. 2012-245092).

Meanwhile, in biopsy needle manipulation, the biopsy needle is inserted with particular attention given to the tip of the needle in the ultrasonic image so as to bypass blood vessels, for example. Thus it has been desired to improve the visibility of the tip of the biopsy needle in the ultrasonic image.

### BRIEF DESCRIPTION OF THE INVENTION

An ultrasonic diagnostic device is provided. The ultrasonic diagnostic device includes an ultrasonic probe which acquires an echo signal by transmitting and receiving ultrasonic waves to and from a test object in a three-dimensional space, a probe identification part which identifies information about the position and orientation of the ultrasonic probe in the three-dimensional space, a biopsy needle identification part which identifies information about the position and orientation, the three-dimensional space, of a biopsy needle to be inserted into the test object; and a control part which controls at least either of two processes, one of the two processes being the transmission and reception of the ultrasonic waves, the other process being data processing based on the echo signal, in accordance with positional relations between the ultrasonic probe and the biopsy needle in the three-dimensional space, the positional relations being identified by the information from the probe identification part and from the biopsy needle identification part.

According to the above aspect, at least either the transmission and reception of the ultrasonic waves, or the data processing based on the echo signal, is controlled in accordance with the positional relations between the ultrasonic probe and the biopsy needle. This can improve the visibility of the biopsy needle.

### BRIEF DESCRIPTION OF THE DRAWINGS

FIG. 1 is a block diagram showing an exemplary overall configuration of an ultrasonic diagnostic device.

FIG. 2 is a block diagram showing a configuration of a display control part of the ultrasonic diagnostic device indicated in FIG. 1.

FIG. 3 is a block diagram showing a configuration of a control part of the ultrasonic diagnostic device indicated in FIG. 1.

FIG. 4 is a diagram for explaining a beam of ultrasonic waves transmitted and received to and from a test object.

FIG. 5 is another diagram for explaining a beam of ultrasonic waves transmitted and received to and from the test object, the diagram showing the biopsy needle inserted into a position deeper than that indicated in FIG. 3.

FIG. 6 is a graphic diagram showing relations between depths in the test object on the one hand and the gains of an echo signal coming from these depths on the other hand.

FIG. 7 is a diagram for explaining an image smoothing process.

FIG. 8 is a block diagram showing another exemplary overall configuration of the ultrasonic diagnostic device.

### DETAILED DESCRIPTION OF THE INVENTION

Exemplary embodiments are explained below.

### First Embodiment

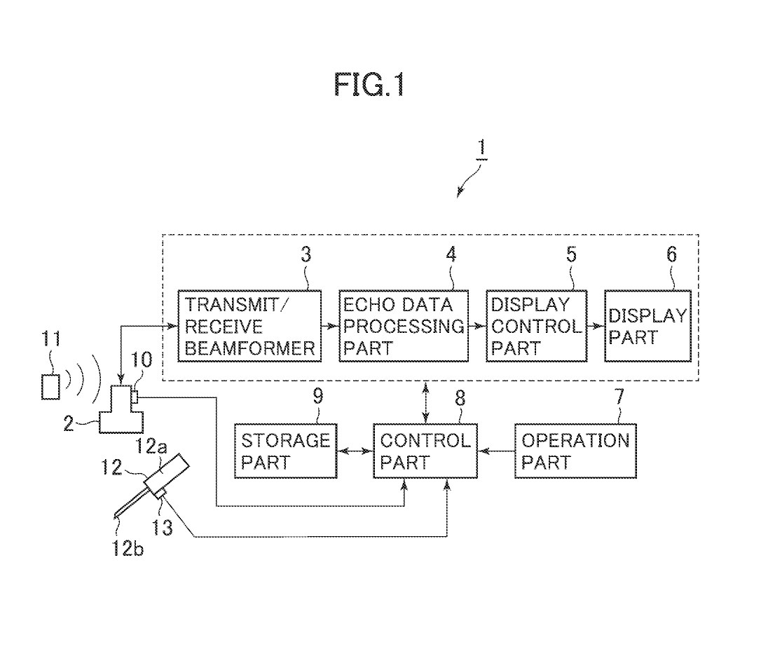

A first exemplary embodiment is explained first. An ultrasonic diagnostic device 1 shown in FIG. 1 includes an ultrasonic probe 2, a transmit/receive beamformer 3, an echo data processing part 4, a display control part 5, a display part 6, an operation part 7, a control part 8, and a storage part 9. The transmit/receive beamformer 3, echo data processing part 4, display control part 5, display part 6, operation part 7, control part 8, and storage part 9 are installed in the ultrasonic diagnostic device 1 proper. The device proper and the ultrasonic probe 2 are interconnected via a cable.

The ultrasonic probe 2 is configured to have a plurality of ultrasonic transducers (not shown) arranged in an array. The ultrasonic transducers transmit ultrasonic waves to the test object and receive an echo signal therefrom. The ultrasonic probe 2 is an execution example of the ultrasonic probe.

The ultrasonic probe 2 is provided with the first magnetic sensor 10 made of a Hall element, for example. The first magnetic sensor 10 detects magnetism generated by a magnetism generation part 11 made of a magnetism generation coil, for example. The magnetism generated by the magnetism generation part 11 forms a coordinate system in a three-dimensional space.

A detection signal from the first magnetic sensor 10 is input to the control part 8. The magnetism generation part 11 and the first magnetic sensor 10 are provided to detect the position and tilt of the ultrasonic probe 2, as will be discussed later.

The first magnetic sensor 10 is an execution example of the first magnetic sensor according. Also, the magnetism generation part 11 is an execution example of the magnetism generation part.

The transmit/receive beamformer 3 supplies the ultrasonic probe 2 with an electric signal for forming a transmission beam of ultrasonic waves based on predetermined transmission parameters, the electric signal being based on a control signal from the control part 8. The transmit/receive beamformer 3 performs signal processing such as signal amplification with a predetermined gain, AD conversion, and additional phasing on the echo signal received by the ultrasonic probe 2, thereby forming the ultrasonic transmission beam with the predetermined trans-mission parameters.

For example, as will be discussed later, the transmit/receive beamformer 3 adjusts the beam direction (sound ray direction) of a transmission and reception beam of ultrasonic waves as well as the focus of the transmission and reception beam based on the control signal from the control part 8.

Given echo data that is output from the transmit/receive beamformer 3, the echo data processing part 4 performs processing to generate an ultrasonic image. For example, the echo data processing part 4 performs B-mode processing such as logarithmic compression and envelope detection to generate B-mode data.

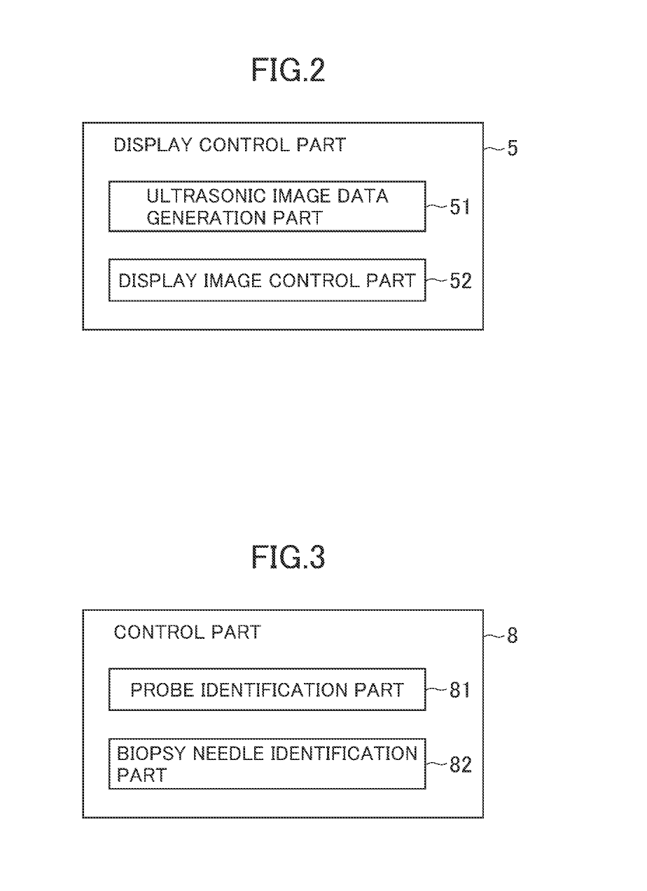

As shown in FIG. 2, the display control part 5 has an ultrasonic image data generation part 51 and a display image control part 52. The ultrasonic image data generation part 51 generates ultrasonic image data using a scan converter to scan convert the data (raw data) input from the echo data processing part 4. The ultrasonic image data generation part 51 generates B-mode image data based on the B-mode data, for example.

The display image control part 52 causes the display part 6 to display an ultrasonic image based on the ultrasonic image data. The ultrasonic image is a B-mode image, for example.

The display part 6 is an LCD (Liquid Crystal Display), an organic EL (electro-Luminescence) display or the like.

The operation part 7 is configured to include a keyboard used by an operator to input instructions and information, and a pointing device such as a trackball, not shown.

The control part 8 is configured to include a CPU (Central Processing Unit). The control part 8 reads control programs stored in the storage part 9 and thereby causes the diverse parts of the ultrasonic diagnostic device 1 to execute their functions.

Also, as shown in FIG. 3, the control part 8 has a probe identification part 81 that executes a probe identification function for identifying the position and orientation of the ultrasonic probe 2. The control part 8 also has a biopsy needle identification part 82 that executes a biopsy needle identification function for identifying the position and orientation of a biopsy needle 12 (see FIG. 1) to be inserted into the test object.

Based on a magnetism detection signal from the first magnetic sensor 10, the probe identification part 81 calculates information about the position and orientation of the ultrasonic probe 2 in a three-dimensional coordinate system with its origin being the magnetism generation part 11 (the information is called the probe position information hereunder). The probe identification part 81 is an execution example of the probe identification part.

The biopsy needle identification part 82 identifies the position and orientation (coordinates) of the biopsy needle 12 (see FIG. 1) in the three-dimensional coordinate system with its origin being the magnetism generation part 11. More specifically, the biopsy needle 12 is provided with a second magnetic sensor 13 made of a Hall element, for example. The second magnetic sensor 13 is positioned at a predetermined distance “d” from the needle tip of the biopsy needle 12. The second magnetic sensor 13 detects magnetism generated by the magnetism generation part 11. A detection signal from the second magnetic sensor 13 is input to the control part 8. The biopsy needle identification part 82 identifies the position and orientation of the biopsy needle 12 based on the magnetism detection signal from the second magnetic sensor 13. The biopsy needle identification part 82 is an execution example of the biopsy needle identification part.

Here, the biopsy needle 12 has a grip part 12a and a needle part 12b attached to the grip part 12a for insertion in the test object. For example, the position of the needle part 12b is identified as the position of the biopsy needle 12. What follows is a more detailed explanation. First, the position of the second magnetic sensor 13 in the three-dimensional space is identified on the basis of the magnetism detection signal from the second magnetic sensor 13. The positional relations between the second magnetic sensor 13 and the needle part 12b are stored beforehand in the storage part 9. Based on the positional relations and on the magnetism detection signal from the second magnetic sensor 13, the position of the needle part 12b is identified (i.e., from the tip (needle tip) of the needle part 12b to the edge of the grip part 12a).

The biopsy needle identification part 82 is an execution example of the biopsy needle identification part. The biopsy needle identification function above is an execution example of the biopsy needle identification function. Also, the second magnetic sensor 13 is an execution example of the second magnetic sensor.

The control part 8 outputs the control signal to at least one of the parts of the ultrasonic diagnostic device 1, the control signal being one which controls at least either of two processes, one of the two processes being the transmission and reception of ultrasonic waves, the other process being data processing based on the echo signal, in accordance with the positional relations between the ultrasonic probe 2 and the biopsy needle 12 in the three-dimensional space, in such a manner that the visibility of the biopsy needle 12 in the ultrasonic image will be optimized (control function). The positional relations between the ultrasonic probe 2 and the biopsy needle 12 are identified both by the position and orientation of the ultrasonic probe 2 identified by the probe identification part 81 and by the position and orientation of the biopsy needle 12 identified by the biopsy needle identification part 82.

In this context, the term “optimization” means bringing about the best visibility of the biopsy needle 12 in the ultrasonic image with various conditions taken into consideration. With this embodiment, the beam direction and the focus of the transmission and reception beam of ultrasonic waves are controlled on the basis of the position and orientation of the biopsy needle 12, which will be discussed later in more detail. The control part 8 is an execution example of the control part.

The storage part 9 is an HDD (Hard Disk Drive) or a semiconductor memory such as a RAM (Random Access Memory) or a ROM (Read Only Memory).

The workings of the ultrasonic diagnostic device 1 of this embodiment will now be explained. First, the operator causes the ultrasonic probe 2 in contact with the test object's body surface to transmit and receive ultrasonic waves to and from the test object, thereby getting an ultrasonic image displayed on the display part 6. It is assumed here that a B-mode image is displayed. The operator then inserts the biopsy needle 12 into the test object along the transmitting and receiving surface of the ultrasonic waves. This allows the biopsy needle 12 to be displayed in the B-mode image.

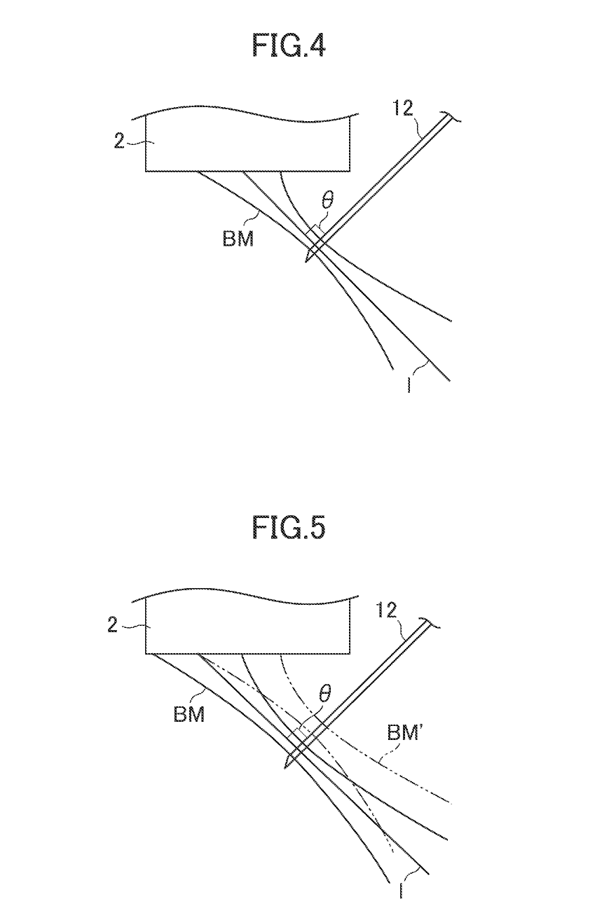

Based on the positional relations between the ultrasonic probe 2 and the biopsy needle 12, the control part 8 outputs the control signal to the transmit/receive beamformer 3 so that, as shown in FIG. 4, the angle θ between an ultrasonic transmission and reception beam BM and the biopsy needle 12 becomes 90 degrees and that the focus (not shown) of the transmission and reception beam BM is on or close to the position of the biopsy needle 12.

The probe identification part 81 identifies the position and orientation of the ultrasonic probe 2 in the three-dimensional space, and the biopsy needle identification part 82 identifies the position and orientation of the biopsy needle in the three-dimensional space, so that the positional relations of the biopsy needle 12 to the ultrasonic probe 2 are identified. Thus on the basis of the positional relations of the biopsy needle 12 to the ultrasonic probe 2, the control part 8 outputs the control signal to the transmit/receive beamformer 3 so that the angle θ between the ultrasonic transmission and reception beam BM and the biopsy needle 12 will become 90 degrees and that the focus (not shown) of the transmission and reception beam BM will be on or close to the position of the biopsy needle 12.

FIG. 4 shows the transmission and reception beam BM of a sound ray 1 passing near the needle tip of the biopsy needle 12 as a transmission and reception beam formed by the transmit/receive beamformer 3. The transmit/receive beamformer 3 also forms transmission and reception beams of multiple sound rays in addition to the transmission and reception beam of the sound ray 1 illustrated. The angle θ of each of these transmission and reception beams is also 90 degrees, and the focus of each of them is on or near the position of the biopsy needle 12 as well.

If the angle θ does not become 90 degrees due to the positional relations between the biopsy needle 12 and the ultrasonic probe 2 for example, the control part 8 outputs the control signal to the transmit/receive beamformer 3 so that the angle θ will become closest to 90 degrees.

Even when the biopsy needle 12 is inserted further into the test object to make the position of the needle tip deeper as shown in FIG. 5, the control part 8 outputs the control signal to the transmit/receive beamformer 3 so that the angle θ between the ultrasonic transmission and reception beam BM and the biopsy needle 12 will become 90 degrees and that the focus (not shown) of the transmission and reception beam BM will be on or close to the position of the biopsy needle 12, on the basis of the positional relations of the biopsy needle 12 to the ultrasonic probe 2. Incidentally, an ultrasonic beam BM′ indicated in FIG. 5 by a two-dot chain line is the ultrasonic beam shown in FIG. 4.

According to this embodiment, based on the positional relations between the ultrasonic probe 2 and the biopsy needle 12, control is performed so that the transmission and reception beam of ultrasonic waves will become perpendicular to the biopsy needle 12 inserted into the test body and that the focus of the transmission and reception beam will be on or close to the position of the biopsy needle 12. As a result, even if the operator does not make input to the operation part 7 to adjust transmission and reception parameters, these parameters are automatically adjusted so that the visibility of the biopsy needle 12 in the B-mode image will be improved.

Although the foregoing paragraphs explained that the transmission and reception beam is controlled based on the positional relations between the ultrasonic probe 2 and the biopsy needle 12, at least a reception beam alone may be controlled instead. That is, the control part 8 may output the control signal to the transmit/receive beamformer 3 so that the angle θ between the reception beam of ultrasonic waves and the biopsy needle 12 will become 90 degrees and that the focus (not shown) of the reception beam will be on or close to the position of the biopsy needle 12.

As another alternative, the control part 8 may output the control signal to the transmit/receive beamformer 3 so that the angle θ between the transmission and reception beam of ultrasonic waves and the biopsy needle 12 will become 90 degrees only near the needle tip of the biopsy needle 12 and that the focus (not shown) of the transmission and reception beam will be on or near the position of the biopsy needle 12.

### Second Embodiment

A second exemplary embodiment is explained next. Only the differences from the first embodiment will be discussed below.

With the second embodiment, the transmit/receive beamformer 3 adjusts the center frequency of the ultrasonic waves transmitted in accordance with the control signal from the control part 8. The center frequency of the transmitted ultrasonic waves is adjusted based on the positional relations between the ultrasonic probe 2 and the biopsy needle 12. Specifically, the control part 8 outputs the control signal to the transmit/receive beamformer 3 so that the closer the needle tip of the biopsy needle 12 is to the ultrasonic probe 2 and the closer the needle tip is to the body surface (shallow in the test object), the higher the center frequency of the transmitted ultrasonic waves is made.

On the other hand, the control part 8 outputs the control signal to the transmit/receive beamformer 3 so that the farther the needle tip of the biopsy needle 12 is from the ultrasonic probe 2 and the farther the needle tip is from the body surface (deep in the test object), the lower the center frequency of the transmitted ultrasonic waves is made.

According to the second embodiment, the center frequency of the transmitted ultrasonic waves is made higher, the closer the biopsy needle 12 is positioned to the ultrasonic probe 2. This can improve the resolution of those areas in the B-mode image that are close to the body surface. On the other hand, the farther the needle tip of the biopsy needle 12 is positioned from the ultrasonic probe 2, the lower the center frequency of the transmitted ultrasonic waves is made, which provides a B-mode image of high penetration. Thus the visibility of the needle tip of the biopsy needle 12 can be improved in a manner ranging from shallow to deep regions of the test object.

### Third Embodiment

A third exemplary embodiment is explained next. Only the differences from the first and the second embodiments will be discussed below.

With the third embodiment, the transmit/receive beamformer 3 adjusts the gain of the echo signal in accordance with the control signal from the control part 8. The gain is adjusted based on the positional relations between the ultrasonic probe 2 and the biopsy needle 12. Specifically, on the basis of the position of the needle tip of the biopsy needle 12 relative to the ultrasonic probe 2, the control part 8 outputs the control signal to the transmit/receiver beamformer 3 so that the gain of the echo signal from a region reached by the needle tip becomes larger than the gain in effect before the needle tip reaches that region.

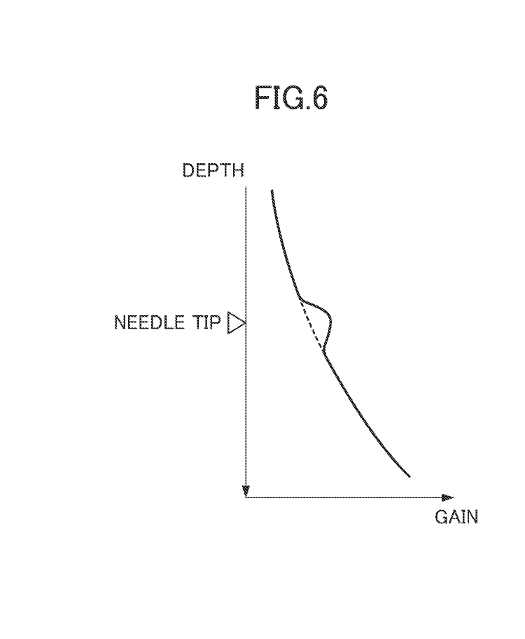

For example, FIG. 6 gives a graph G showing the relations between depths in the test body (distances from the ultrasonic probe 2) on the one hand and the gains of the echo signal from these depths on the other hand. If it is assumed that a triangle illustrated in FIG. 6 represents the position of the needle tip of the biopsy needle 12, the gain of the echo signal from near the needle tip becomes larger than the gain in effect before the needle tip reaches the region, as indicated by broken line.

Incidentally, the depth increases downward along the vertical axis and the gain rises rightward along the horizontal axis in the graph of FIG. 6.

According to the third embodiment, on the basis of the position of the biopsy needle 12 relative to the ultrasonic probe 2, control is performed in such a manner that the gain of the echo signal from near the needle tip of the biopsy needle 12 will become higher than the gain in effect before the needle tip reaches the region. Thus even if the operator does not make input to the operation part 7 to adjust the gain, the gain is automatically adjusted to enhance the visibility of the needle tip of the biopsy needle 12 in the B-mode wave image.

### Fourth Embodiment

A fourth exemplary embodiment is explained next. Only the differences from the first, the second, and the third embodiments will be discussed below.

With the fourth embodiment, the ultrasonic image data generation part 51 performs an image smoothing process on B-mode image data. On the basis of the positional relations between the ultrasonic probe 2 and the biopsy needle 12, the ultrasonic image data generation part 51 identifies the position and orientation that are subject to the image smoothing process so as to optimize the visibility of the biopsy needle 12 in the B-mode image. Given the control signal from the control part 8, the ultrasonic image data generation part 51 carries out the image smoothing process accordingly. The ultrasonic image data generation part 51 is an execution example of the processing part.

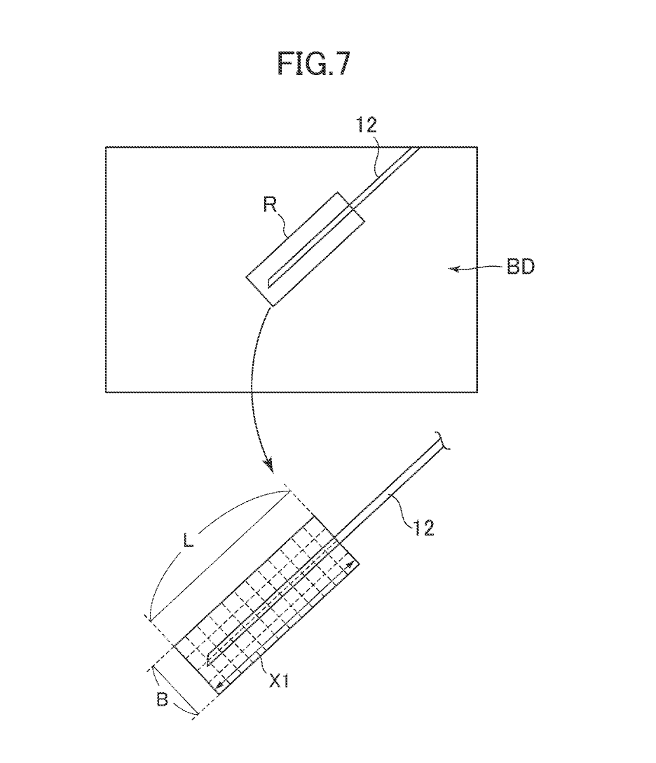

What follows is a more specific explanation of the image smoothing process of the fourth embodiment. As shown in FIG. 7, the ultrasonic image data generation part 51 performs the image smoothing process on each of the B-mode image data BD (i.e., data corresponding to the pixels) in a rectangular region R. It should be noted that in FIG. 7, the biopsy needle 12 is shown amid the B-mode image data BD for purpose of explanation.

The region R has a length L in a direction X1 parallel to the biopsy needle 12 and a predetermined breadth B in a direction X2 perpendicular to the direction X1. The region R has the breadth B centering on the biopsy needle 12. Also, the edge of the region R in its longitudinal direction (direction X1 parallel to the biopsy needle 12) has a predetermined margin relative to the needle tip of the biopsy needle 12. The region R is established based on the positional relations of the biopsy needle 12 to the ultrasonic probe 2.

The ultrasonic image data generation part 51 performs the image smoothing process among the B-mode image data corresponding to the pixels arrayed in the direction X1 of the biopsy needle 12. This can improve the visibility of the biopsy needle 12. In FIG. 7, partitioned quadrangles in broken lines in the region R represent the pixels.

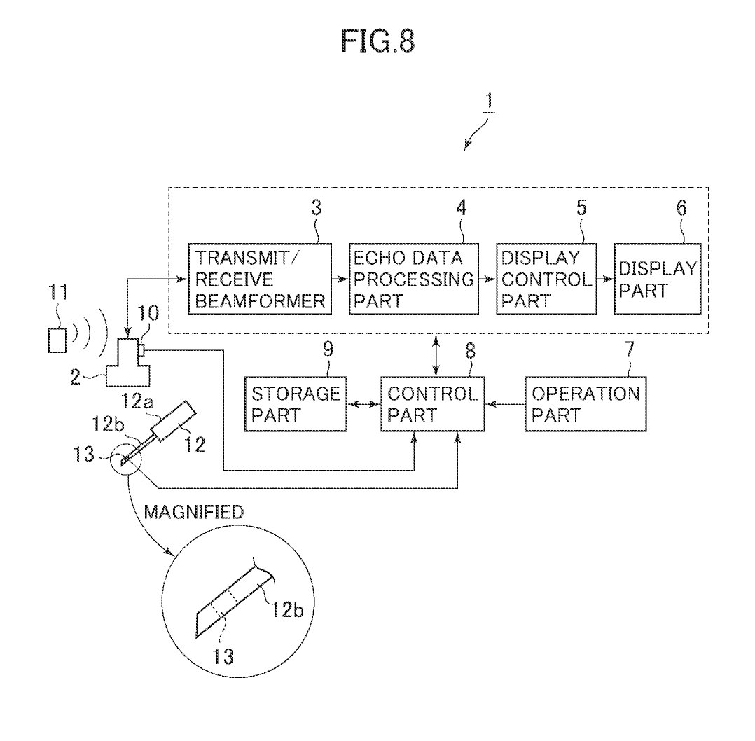

According to the fourth embodiment, the positional relations of the biopsy needle 12 to the ultrasonic probe 2 are identified, and the region R is established to include the biopsy needle 12. The image smoothing process is performed on that region R in the direction X1 parallel to the biopsy needle 12 and in the direction X2 perpendicular to the direction X1. This can improve the visibility of the biopsy needle 12 in the B-mode image.

Whereas the disclosure has been explained above using some exemplary embodiments and execution examples, these are not limitative. It is evident that various modifications, variations and alternatives may be made so far as they are within the scope of the appended claims or the equivalents thereof. For example, the second magnetic sensor 13 may be attached to the needle tip of the biopsy needle 12, as shown in FIG. 8. With the second magnetic sensor 13 fixed to the needle tip of the biopsy needle 12, even if the needle part 10b is bent inside the test object, the biopsy needle identification part 82 can accurately identify the position of the needle part 10b. Thus the transmission and reception of ultrasonic waves and the processing of data based on the echo signal are controlled in accordance with the accurate position information, so that the visibility of the biopsy needle can be improved unfailingly.

As another example, all controls based on the positional relations between the ultrasonic probe 2 and the biopsy needle 12 as explained in conjunction with the first through the fourth embodiments above may be carried out.

As a further example, arrangements can be made to switch between two modes, one mode being such that control is performed based on the positional relations between the ultrasonic probe 2 and the biopsy needle 12 as explained in conjunction with the first through the fourth embodiments above, the other mode being such that control is not performed on the basis of the positional relations between the ultrasonic probe 2 and the biopsy needle 12.

1. An ultrasonic diagnostic device comprising:
an ultrasonic probe configured to acquire an echo signal by transmitting and receiving ultrasonic waves to and from a test object in a three-dimensional space;
a probe identification part configured to identify information about a position and orientation of the ultrasonic probe in the three-dimensional space;
a biopsy needle identification part configured to identify information about a position and orientation, in the three-dimensional space, of a biopsy needle to be inserted into the test object; and
a control part which controls at least one of two processes based on positional relations between the ultrasonic probe and the biopsy needle in the three-dimensional space, the positional relations identified by the information from the probe identification part and the information from the biopsy needle identification part, wherein one of the two messes is the transmission and reception of the ultrasonic waves, and the other of the two processes is data processing based on the echo signal.

2. The ultrasonic diagnostic device according to claim 1, further comprising:
a magnetism generation part installed in the three-dimensional space;
a first magnetic sensor attached to the ultrasonic probe and configured to detect magnetism of the magnetism generation part; and
a second magnetic sensor attached to the biopsy needle and configured to detect magnetism of the magnetism generation part;
wherein the probe identification part is configured to identify the information about the position and orientation of the ultrasonic probe in the three-dimensional space based on a magnetism detection signal from the first magnetic sensor, and
wherein the biopsy needle identification part is configured to identify the information about the position and orientation of the biopsy needle in the three-dimensional space based on a magnetism detection signal from the second magnetic sensor.

3. The ultrasonic diagnostic device according to claim 1, wherein the control part is configured to perform control such that an angle between a reception beam of the ultrasonic waves and the biopsy needle is approximately 90 degrees.

4. The ultrasonic diagnostic device according to claim 2, wherein the control part is configured to perform control such that an angle between a reception beam of the ultrasonic waves and the biopsy needle is approximately 90 degrees.

5. The ultrasonic diagnostic device according to claim 1, wherein the control part is configured to perform control such that a reception beam of the ultrasonic waves is focused on the biopsy needle.

6. The ultrasonic diagnostic device according to claim 2, wherein the control part is configured to perform control such that a reception beam of the ultrasonic waves is focused on the biopsy needle.

7. The ultrasonic diagnostic device according to claim 3, wherein the control part is configured to perform control such that a reception beam of the ultrasonic waves is focused on the biopsy needle.

8. The ultrasonic diagnostic device according to claim 1, wherein the control part is configured to perform control such that the farther a needle tip of the biopsy needle is from the ultrasonic probe, the lower a center frequency of the ultrasonic waves transmitted from the ultrasonic probe.

9. The ultrasonic diagnostic device according to claim 2, wherein the control part is configured to perform control such that the farther a needle tip of the biopsy needle is from the ultrasonic probe, the lower a center frequency of the ultrasonic waves transmitted from the ultrasonic probe.

10. The ultrasonic diagnostic device according to claim 5, wherein the control part is configured to perform control such that the farther a needle tip of the biopsy needle is from the ultrasonic probe, the lower a center frequency of the ultrasonic waves transmitted from the ultrasonic probe.

11. The ultrasonic diagnostic device according to claim 6, wherein the control part is configured to perform control such that the farther a needle tip of the biopsy needle is from the ultrasonic probe, the lower a center frequency of the ultrasonic waves transmitted from the ultrasonic probe.

12. The ultrasonic diagnostic device according to claim 1, wherein the control part is configured to perform control such that a gain of the echo signal from a region reached by a needle tip becomes larger than a gain in effect before the needle tip reaches the region.

13. The ultrasonic diagnostic device according to claim 2, wherein the control part is configured to perform control such that a gain of the echo signal from a region reached by a needle tip becomes larger than a gain in effect before the needle tip reaches the region.

14. The ultrasonic diagnostic device according to claim 5, wherein the control part is configured to perform control such that a gain of the echo signal from a region reached by a needle tip becomes larger than a gain in effect before the needle tip reaches the region.

15. The ultrasonic diagnostic device according to claim 6, wherein the control part is configured to perform control such that a gain of the echo signal from a region reached by a needle tip becomes larger than a gain in effect before the needle tip reaches the region.

16. The ultrasonic diagnostic device according to claim 1, further comprising a processing part configured to smooth an ultrasonic image of the test object by performing data processing based on the echo signal;
wherein the control part is configured to control the processing part performing the data processing based on the echo signal such that the ultrasonic image is smoothed at or near the biopsy needle in a direction parallel to the biopsy needle.

17. The ultrasonic diagnostic device according to claim 2, further comprising a processing part configured to smooth an ultrasonic image of the test object by performing data processing based on the echo signal;
wherein the control part is configured to control the processing part performing the data processing based on the echo signal such that the ultrasonic image is smoothed at or near the biopsy needle in a direction parallel to the biopsy needle.

18. The ultrasonic diagnostic device according to claim 2, wherein the second magnetic sensor is attached to a needle tip of the biopsy needle.

19. The ultrasonic diagnostic device according to claim 2, wherein the second magnetic sensor is positioned a predetermined distance from a needle tip of the biopsy needle.

20. An ultrasonic diagnostic device configured to cause a computer to implement:
a probe identification function which identifies information about a position and orientation, in a three-dimensional space, of an ultrasonic probe for acquiring an echo signal by transmitting and receiving ultrasonic waves to and from a test object in the three-dimensional space;
a biopsy needle identification function which identifies information about a position and orientation, in the three-dimensional space, of a biopsy needle to be inserted into the test object; and
a control function which controls at least one of two processes based with positional relations between the ultrasonic probe and the biopsy needle in the three-dimensional space, the positional relations identified by the information from the probe identification function and the information from the biopsy needle identification function, wherein one of the two processes is the transmission and reception of the ultrasonic waves, and the other of the two processes is data processing based on the echo signal.
